(19) United States
(12) Patent Application Publication
(10) Pub. No.: US 2017/0188950 A1
(43) Pub. Date: Jul. 6, 2017

(54) SHOE INSERT FOR MONITORING OF BIOMECHANICS AND MOTION

(71) Applicant: Motion Metrix Corporation, Half Moon Bay, CA (US)

(72) Inventors: Bence Gazdag, Half Moon Bay, CA (US); Bruce White, Santa Clara, CA (US); Marc Boutillette, Olean, NY (US); Robert Csakvari, Budapest (HU)

(21) Appl. No.: 15/394,407

(22) Filed: Dec. 29, 2016

Related U.S. Application Data
(60) Provisional application No. 62/273,091, filed on Dec. 30, 2015.

(51) Int. Cl. A61B 5/00; A61B 5/103; A43B 17/14; A43B 3/00; A43B 17/00

(52) U.S. Cl. CPC A61B 5/6807; A43B 3/0005; A43B 17/006; A43B 17/14; A61B 5/1038; A61B 5/0022; A61B 5/742; A61B 5/7475; A61B 2562/0219; A61B 2560/0475; A61B 2562/0247

(57) ABSTRACT

Systems and methods for a self-contained shoe insole device to monitor biomechanics and motion are disclosed. The systems and methods allow monitoring for orthopedic diagnostics, fitness tracking, and social/gaming activities using a shoe insole device with multiple sensor locations for pressure, acceleration, rotation rate, all forms of inertial data in three axes, position/location, heart rate, and other physical attributes. The shoe insole device can include a plurality of layers, with one layer containing a plurality of sensors, and an electronics component for collecting, reading, storing and transmitting the sensor data. The shoe insole device can wirelessly connect with external computing devices for monitoring and feedback directly to the user or a health care or fitness training professional, or across multiple users in a social or gaming situation. The system can further be provided for monitoring and tracking physical activity and enable a variety of interactions based upon the collected data.

### CROSS-REFERENCE TO RELATED APPLICATIONS

This application claims priority to U.S. Provisional Patent Application Ser. No. 62/273,091, filed on Dec. 30, 2015, to Bence Gazdag et al., entitled “Shoe Insert for Monitoring of Biomechanics and Motion,” currently pending, the entire disclosure of which is incorporated herein by reference.

### FIELD OF THE INVENTION

The present invention relates to a device for monitoring biomechanics and motion for orthopedic, athletic, fitness, health and social uses. In particular, the present invention relates to a self-contained shoe insole for monitoring biomechanics and motion, and to systems and methods for using a self-contained shoe insole to monitor biomechanics and motion.

### BACKGROUND OF THE INVENTION

Basic wearable diagnostic technology has been in existence since at least 1977, pioneered by Polar which started with heart rate monitors for use while exercising. Many other companies including Garmin, Adidas and Timex brought products to the market which measured heart rate, though all of these products had to be connected to computers or other devices through wires to access the data. Pedometers also have long historical use but were not common to the fitness industry as the accuracy was low, devices were comparatively large, and transferring data from the devices was not easy.

The convergence of micro sensor, mobile and Bluetooth technologies in the early 2000s enabled the possibilities for monitoring much more than just heart rate and basic step count. Many large and small companies have entered the market with wearable products that measure a wide variety of data points. Companies such as Polar and Garmin specialize in heart rate and global position system (GPS) measurements while other companies such as Jawbone, FitBit and Nike specialize in using micro-sensors to calculate movement. Most of these companies focus on products that are worn on the wrist (watches and bands) or that clip to a piece of clothing or sit in a pocket.

All of these wearable products have accompanying mobile and web applications which provide the user information about the data the device is tracking and allows the user to log a history of their movement data. These applications also offer APIs to allow other mobile applications to use the data gathered by the wearable device and provide their own services, an example of this is the RunKeeper mobile application that uses data from the Jawbone UP product. While these independent applications offer some similar functionality to our application they are lacking key features and functionality.

Recently there have been some entries into the market for shoe insoles that use micro-sensors. Moticon has a wired version of a micro-sensor enabled insole which tracks pressure and Sensoria has a micro-sensor enabled sock which tracks pressure. There are also several diagnostic shoe insoles that connect through wires to large power and data interpretation pieces (usually strapped to the leg) that are sold commercially to physical fitness and physician's offices.

Specifically, known prior art tracking at the foot includes the Surrosense RX system, the Zybeimind Achillex, the Lechal Shoe, the Geopalz iBitz, and the Boogio. Several other wearable activity trackers are known in the art which do not track at the foot, but rather focus on general movement, heartrate, and sleep tracking. Within the foot tracking art, no system allows for embedding the entire device as an interchangeable unit within the shoe, and none includes the combined measures of pressure, acceleration, rotation rate and all forms of inertial data in three axes, GPS tracking, social uses (such as gamification around fitness metrics), and large-scale biomechanical diagnostic monitoring. Thus, a consumer-level insole which provides integrated biomechanical diagnostics with physical activity tracking through a wireless connection on a mobile application is absent from the current art.

### BRIEF SUMMARY OF THE INVENTION

The present invention is directed toward a system for monitoring and tracking biomechanics and other data from one or more users. The system can be a wireless, monitored, wearable system and can include a wearable shoe insole device, an electronics component connected to the shoe insole device and an external computer system. As described herein, the wearable shoe insole device can be configured to monitor pressure, acceleration, rotation rate and all forms of inertial data in three axes, and route tracking of a user, and then transmit the collected data to the external computer system for feedback on orthopedic metrics and fitness monitoring. The wireless, monitored, wearable system can also enable social and gaming activities related to fitness metrics and large-scale monitoring of orthopedic metrics for diagnostic and prevention recommendations.

The shoe insole device can include multiple layers, including at least one sensor layer. The sensor layer(s) can include a plurality of sensors positioned and distributed across the foot-bed of the sensor layer. The plurality of sensors can include a variety of different types of sensors for collecting pressure data, acceleration data, temperature data, rotation data, inertial data in three axes, route tracking data, position data and other monitoring data.

The insole device can also include an electronics component, which can be contained within a heel cup layer of the insole device according to certain embodiments of the present invention. The electronics component can be configured to collect the data received by the plurality of sensors in the sensor layer. In addition, the electronics component can be configured with certain sensors that do not need to be positioned along the foot-bed (e.g., GPS sensors, acceleration sensors). The electronics component can include one or more processors for receiving and processing the sensor data, a battery for powering the electronics component and insole device, a memory component for storing the sensor data, and a transmitting component for wirelessly transmitting the collected sensor data.

The shoe insole device (and the electronics component) can be wirelessly connected to a computer system, such as a mobile device with a mobile application, which can provide a display and/or user interface for configuring and displaying the collected sensor data. The computer system can also include one or more processors, memory components, and machine readable instructions for examining, parsing, and configuring the collected sensor data for display and use to one or more users. The computer system can further be configured to transmit the collected sensor data to a remote server to allow for aggregation and/or analysis of the collected sensor data of several different users.

The present invention is further directed toward a method for using the wearable insole device and system. The method can include the steps of (i) collecting sensor data from the plurality of sensors contained within the insole device sensor layer and connected to the electronics component, (ii) storing the collected sensor data in the memory of the electronics component, (iii) transmitting the collected sensor data to an external computer system via a wireless network, (iv) examining, parsing and configuring the collected sensor data through a series of instructions contained on the external computer system, and (v) displaying the configured sensor data through a user interface provided on the external computer system.

Other aspects and advantages of the present invention will be apparent from the following detailed description of the preferred embodiments and the accompanying drawing figures.

### DESCRIPTION OF THE SEVERAL VIEWS OF THE DRAWING

In the accompanying drawing, which forms a part of the specification and is to be read in conjunction therewith in which like reference numerals are used to indicate like or similar parts in the various views:

FIG. 1 is a block diagram illustrating a network-based computer system for operating and interacting with a monitoring shoe insole device in accordance with one embodiment of the present invention;

FIG. 2 is a block diagram illustrating an electronic computer component for a monitoring shoe insole device in accordance with one embodiment of the present invention;

FIG. 3 is a flowchart illustrating a method of operating an computer component of a shoe insole device in accordance with one embodiment of the present invention;

FIG. 4 is a perspective view of a shoe insole device in accordance with one embodiment of the present invention;

FIG. 4A is an exploded perspective view of the shoe insole device of FIG. 4 in accordance with one embodiment of the present invention; and

FIG. 5 is a schematic diagram of a graphical user interface for use in connection with a shoe insole device in accordance with one embodiment of the present invention.

### DETAILED DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

Some portions of the detailed descriptions which follow are presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, terms such as “processing” or “computing” or “calculating” or “determining” or “displaying” or the like, refer to the action and processes of a computer system, or similar computing device, that manipulates and transforms data represented as physical (e.g., electronic) quantities within the computer system's registers and memories into other data similarly represented as physical quantities within the computer system memories or registers or other such information storage, transmission or display devices.

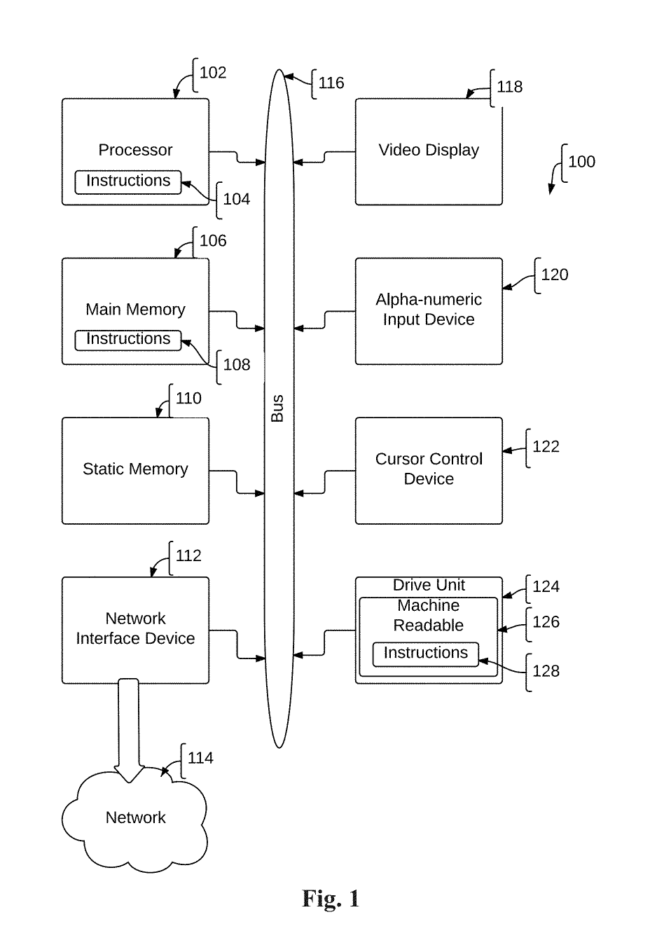

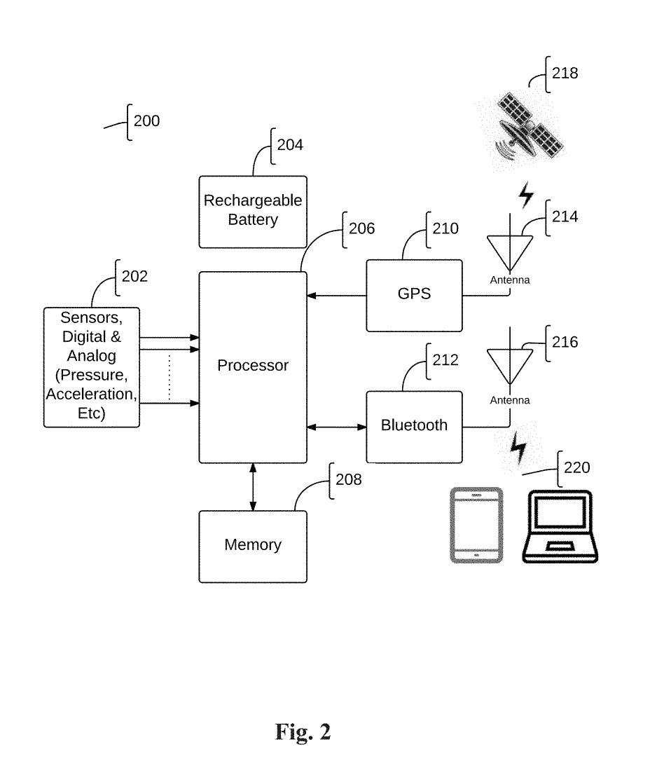

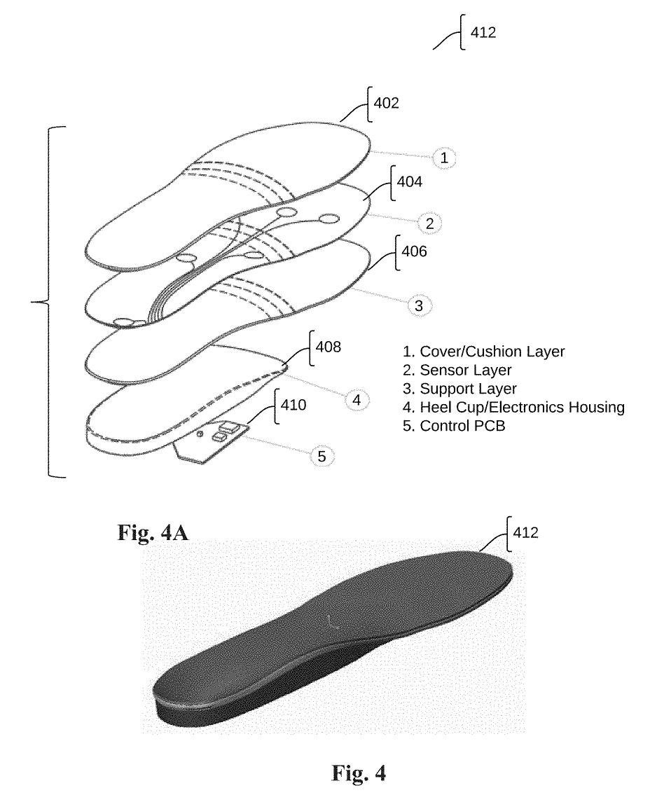

The present invention is directed generally toward a system for monitoring and tracking biomechanics and other data from a user. The system can comprise a monitoring shoe insole 412, an electronic component 200 and an external computer system and/or software 100. The monitoring shoe insole 412, as illustrated in FIG. 4, can be for monitoring and tracking pressure, acceleration, rotation rate and all forms of inertial data in three axes, location and other data and info′ nation. The electronic component 200, illustrated schematically in FIG. 2, can be located within or coupled to monitoring shoe insole 412 and can be used to track, collect, compile and relay various data and information as described in greater detail below. The electronic component 200 can be embedded within shoe insole 412, or can be otherwise attached or positioned adjacent to shoe insole 412. Shoe insole 412 can be used in connection with the external computer system and/or software 100, as schematically illustrated in FIG. 1 and described in greater detail below. Computer system 100 can be in the form of a smartphone, stand-alone electronic device or other computer system. Shoe insole 412 can be wirelessly connected to and interoperable with computer system 100. For illustrative purposes, the various embodiments described below are with reference to a wirelessly connected, wearable shoe insert 412 for tracking pressure, acceleration, rotation rate and all forms of inertial data in three axes, and location. The most common example described in detail is a shoe insole 412 with embedded electronics 200 and interoperable with a smartphone or other computer system 100. These embodiments and examples of suitable environments are not intended to suggest any limitation as to the scope of use or functionality of the present invention. Accordingly, they should not be interpreted as having any dependency or requirement relating to any one or a combination of the components illustrated in the exemplary operating environments described herein.

The combined shoe insole 412 with electronic component 200 and computer system 100 can be a fully self-contained wireless interchangeable shoe insole 412 interoperable with software on a smartphone or computer system 100. The combination can monitor pressure, acceleration, rotation rate and all forms of inertial data in three axes, and rout tracking and have the ability to provide user feedback on orthopedic metrics including foot pronation, supination, heel strike, general walking/running/bicycling form, and other biomechanical issues; track a complete suite of user fitness metrics, including but not limited to, running/walking/cycling cadence, distance, calories burned, and pulse; enable socialization/gamification of user fitness metrics; and enable data mining of large-sample-size user fitness and orthopedic statistics to discern the subtle causes of pathology and methods of prevention. The shoe insole 412 can be configured to perform all of these functions without being directly wired to a device external to the shoe insole 412, such as by interacting only with a stand-alone smartphone or computer system 100. A smartphone application can be used for displaying the collected shoe insole data (i.e., pressure, acceleration, rotation rate and all forms of inertial data in three axes, GPS data) as described in greater detail below. In alternative embodiments, Shoe insole 412 can require synchronization through the wirelessly connected device to a central data collection service. Such embodiments can be advantageous for allowing for social collaboration based upon the collected data or data analysis using data from multiple users to identify and diagnose biomechanical issues which can be subsequently reported to the user via their wireless device.

In the specifics of discussing the wireless, sensor-enabled shoe insole 412, several definitions will be used in the specification. First, “wireless” can mean any process relating to the transmission of data without a cable. Specifically, the wireless system must include or have the capacity to connect to another electronic device to transfer data in a unidirectional or bidirectional manner. Common art for wireless connections include standards such as Bluetooth®, Wi-Fi™, Wi-MAX, CDMA, 3g, 4g, and numerous other standard and custom or proprietary technologies. Further, “sensor-enabled” can mean any collection of one or more sensors including or having the capacity for direct physical measurements such as temperature, pressure, vibration, pulse, etc., secondary physical attributes such as acceleration (movement), electrical conductance (sweat), work (caloric energy expended), etc., or external/absolute measurements such as location (GPS), time, etc.

FIG. 1 is intended to provide a brief, general description of suitable computer hardware and a suitable computing environment in conjunction with which several different embodiments may be implemented. Some of the embodiments are described in the general context of computer-executable instructions, such as program modules, being executed by a computer 100. Program modules can include routines, programs, objects, components, data structures, etc. that can perform particular tasks or implement particular abstract data types.

The computer system 100 described herein can be spread across many physical hosts so that many systems 100 and/or sub-systems 100 can be used in implementing the operation of the present invention. Computer system 100 can also have several different system configurations, including but not limited to, hand-held devices, multiprocessor systems, microprocessor-based or programmable consumer electronics, network PCs, minicomputers, mainframe computers, and the like. The present invention can also be used with distributed computer environments where tasks are performed by I/O remote processing devices that can be linked through a communications network. In such distributed computer environments, program modules can be located in both local and remote memory storage devices. Collectively, a distributed computer environment can foul′ one embodiment of computer system 100.

Computer system 100 can have a hardware and operating environment that is applicable to both servers and/or remote clients. Computer system 100 can be located within a machine and can have instructions for causing the machine to perform any one or more of the embodiments of the present invention. In certain embodiments, the machine (and computer system) 100 can operate as a stand-alone device or may be connected (e.g., networked) to other machines. In a networked deployment, the machine and system 100 can operate in the capacity of a server or a client machine in a service-client network environment or as a peer machine in a peer-to-peer (or distributed) network environment. While only a single machine is illustrated, the term “machine” shall also be taken to include any collection of machines that individually or jointly execute a set, or multiple sets of instructions to perform any one or more of the methodologies discussed herein.

FIG. 1 illustrates one embodiment of computer system 100 that can be used in connection with shoe insole 412. Computer system 100 can include a processor 102 (e.g., a central processing unit (CPU), a graphics processing unit (GPU) or both), a main memory 106 and a static memory 110, which can communicate with each other via a bus 116. Computer system 100 can further include a video display unit 118 (e.g., a liquid crystal display (LCD) or a cathode ray tube (CRT)). Video display unit 118 can also comprise any other suitable graphical user interface. In certain embodiments of the present invention, computer system 100 can also include one or more of an alpha-numeric input device 120 (e.g., a keyboard), a user interface (UI) navigation device or cursor control device 122 (e.g., a mouse, a touch screen), a disk drive unit 124, a signal generation device (e.g., a speaker), and a network interface device 112.

Disk drive unit 124 can include a machine-readable medium 126 that can store one or more sets of instructions 128. Instructions 128 can include data structures, such as software instructions, that embody any one or more of the methodologies or functions described herein. Instructions 128 can also reside, completely or at least partially, within the main memory 108 or within the processor 104 during execution thereof by the computer system 100. In such an embodiment, main memory 106 and processor 102 also constitute machine-readable media. The instructions 128 can allow computer system 100 to compile, organize, filter, and display data collected from the shoe insole 412 via electronic component 200.

While the machine-readable medium 126 is illustrated in FIG. 1 as a single medium, the term “machine-readable medium” incorporates both a single medium and multiple media (e.g., a centralized or distributed database, or associated caches and servers) that store the one or more instructions 128. The term “machine-readable storage medium” also includes any tangible medium that is capable of storing, encoding, or carrying instructions 128 for execution by computer system or machine 100 causing computer system or machine 100 to perform operations and methodologies of the present invention, or that is capable of storing, encoding, or carrying data structures used by or associated with such instructions 128. The term “machine-readable storage medium” includes, but is not limited to, solid-state memories and optical and magnetic media that can store information in a non-transitory manner (i.e., media that is able to store information for a period of time, however brief). Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks.

Instructions 128 can be transmitted or received over a communications network 114 using a transmission medium via network interface device 112 and utilizing any one of a number of well-known transfer protocols (e.g., FTP, HTTP). Communication network 114 can be a local area network (LAN), a wide area network (WAN), the Internet, a mobile telephone network, a Plain Old Telephone (POTS) network, a wireless data network (e.g., WiFi and WiMax networks), as well as any proprietary electronic communications systems that might be used. The term “transmission medium” includes any intangible medium that is capable of storing, encoding, or carrying instructions for execution by the machine 100, and includes digital or analog communications signals or other intangible medium to facilitate communication of such software.

In a preferred embodiment, computer system 100 includes operation of the entire system on a remote server with interactions occurring from individual connections over the network 114 to handle user input as an internet application.

FIG. 2 illustrates a block diagram of one embodiment of the electronic component 200 of shoe insole 412. Electronic component 200 can be used in connection with and/or embedded in shoe insole 412. In the diagram shown in FIG. 2, the various subcomponents of electronic component 200 are represented. Electronic component 200 can include a suite of one or more digital and analog sensors 202. Sensors 202 can be pressure sensors, acceleration sensors, temperature sensors, rotation rate sensors and the like. Each sensor can be intercoupled with a processor 206 to provide the processor with sensor data. Processor 206 can be an electrical component with the ability to receive and process input and to provide output. Processor 206 can be a customized electronic device or be a general computing component, similar to processor 102 in computer system 100.

A non-rechargeable or rechargeable battery 204 can be supplied with electronic component 200. Battery can be coupled with each subcomponent depending upon the specific needs of the particular subcomponent. Rechargeable battery 204 can be a chemical battery, such as alkaline, lithium ion, lithium polymer, nickel metal hydride, nickel cadmium, and the like. Rechargeable battery 204 can also be a charge storing capacitor or other electricity storing component. Similarly, Battery 204 can be a mechanical device such as a spring coupled to an electric generator. Further, one of ordinary skill in the art can identify multiple methods which may be used to charge the battery 204, but are not discussed in detail in the present disclosure, including plug-in via direct wire, inductive charging, inertial recapture, or the like.

A GPS sensor 210 can also be coupled with processor 206 as illustrated in FIG. 2. GPS sensor 210 can take input from an array of earth orbital satellites 218 via an antenna 214. The arrangement of the satellites 218 and their encoded signals can allow GPS sensor 210 to determine a physical location of the electronic component's antenna 214, which can be provided to processor 206.

Processor 206 can be intercoupled with a memory 208. The memory 208 can used to store the data collected from sensors 202 and GPS sensor 210. The memory 208 can be written to, or read from and can be any type of machine readable storage device (similar to memory 110 and 124 as illustrated in FIG. 1), including any solid-state memories and optical and magnetic media that can store information in a non-transitory manner (i.e., media that is able to store information for a period of time, however brief). Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; and magneto-optical disks.

Processor 206 can be connected to one or more external electronic devices 220 via component antenna 216. To enable connection with the one or more external electronic devices 220, antenna 216 can be connected to a Bluetooth 212 or other standards-based or proprietary short-range wireless communication system. The Bluetooth 212 connection allows interaction with the processor 206 to control the specific collection or configuration of sensor data 202, 210, to retrieve the sensor data from memory 208, to clear the memory 208 of stored sensor data 202, 210, to register the available capacity of the rechargeable battery 204, or similar data access or device control capabilities.

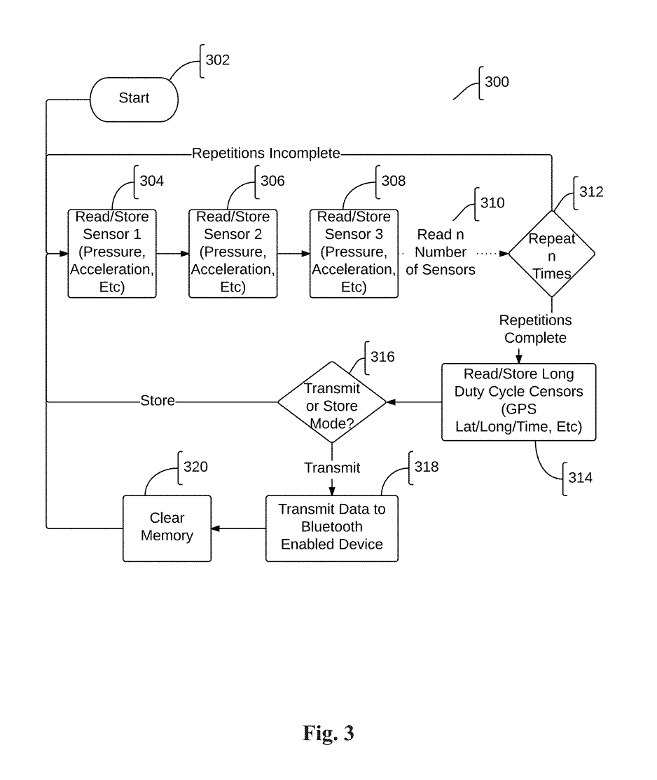

Electronic component 200 can be used to collect various biometric and motion data obtained while shoe insole 412 is in use. FIG. 3 illustrates one particular method of using the shoe insole 412 with electronic component 200 and computer system 100. The method shown in FIG. 3 incorporates performing actions in accordance with measurements and data process flow. As illustrated in FIG. 3, the method can start at step 302 by beginning an iteration cycle carried out by steps 304-312. At step 304, component 200 can begin reading and temporarily storing data from a first sensor 304. The sensor data can be pressure, acceleration, rotation rate and all forms of inertial data in three axes, or any other sensor data. Processing can continue at step 306 by reading and temporarily storing data from a second sensor 306, if one is available. Processing can further continue at step 308 by reading and temporarily storing data from a third sensor 308, if available, and can continue to read and store for any number of interconnected sensors 310. Sensors 304-310 can be used for collecting various types of data, either specifically or collectively. For example, one set of first, second, third, and nth sensors can be used to collect only pressure data, while another set of first, second, third, and nth sensors can be used to collect only acceleration data. Thus, steps 304-310 can be performed for each specific type of sensor set. Alternatively, the set of first, second, third, and nth sensors can be configured to collect data used for both pressure and acceleration. Thus, steps 304-310 are performed for only the single set of sensors.

Once all interconnected sensors 304, 306, 308, and 310 have been read and their data temporarily stored, they may be read again in rapid succession if so desired at step 312. Step 312 represents the miming of one or more iteration cycles begging at step 304. However, if they have been read a sufficient number of times for the desired need, then any sensors that require a longer sampling time can be read at step 314. For example, sensors such as GPS sensors can take seconds or minutes to properly register, and therefore can be read at step 314. The one or more of the long duty cycle sensors 314 can be read similar to the short duty cycle sensors previously read 304-310.

Depending on the current system configuration at the time of operation, the collected sensor data 304-310, 314 can be transmitted or stored at step 316 in local memory 208. If the data is stored in local memory 208, then processing can again returns to collecting initial sensor data 304. Otherwise, if the sensor data is set to transmit, then the data can be transmitted at step 318 to a connected device 212, 220 using a wireless transmission technology such as Bluetooth. Once the data is transmitted, then the local memory 208 can be cleared at step 320 before returning to collecting sensor data 304.

While the described flow chart in FIG. 3 includes an explicit description of repeated actions, the repetition count can be any number including zero repetitions, meaning a single pass through. In addition, only one repetition cycle is shown, but others may exist within sub-groups of sensors. For example, the first sensor at step 304 can be read any number of times before the second sensor at step 306 is read. The combination of first sensor at step 304 and second sensor at step 306 can also be read any number of times before the third sensor at step 308 is read. This generalized repetition of sensor reading can include both the short duty cycle and long duty cycle sensors in any combination. Similarly, the system may allow a combination of transmit and store modes at step 316 where the data can be stored for a certain number of repetitions before transmitting.

FIGS. 4 and 4A illustrate one embodiment of the shoe insole 412. The sensor-embedded insole assembly 412 can be comprised of multiple layers as illustrated in FIG. 4A. Shoe insole 412 can include a top layer 402 that can be a cover or cushion layer constructed of fabric, or foam material, either natural or synthetic, such as wool, cotton, nylon, polyurethane, and the like. Top layer 402 can provide comfort and protection from a sensor layer 404, that can be placed below top layer 402, while still allowing relevant data to pass through to the sensors (such as pressure, temperature, sweat/conductance, etc.).

Shoe insole 412 can also include a sensor layer 404 that can contain a variety of sensors 202 to enable proper data collection. Sensors 202 can be low profile sensors distributed across the foot-bed of sensor layer 404 to measure discrete points on the foot for pressure, temperature, and similar information. Sensor layer 404 can be constructed as a thin polymer sheet (or other suitable material) with sensors 303 embedded therein. Alternatively, sensors 202 can be located on sensor layer 404 but not coupled to any sort of other material. In certain embodiments of the present invention, the shoe insole 412 can include multiple sensor layers or can include sensors positions on or within other layers in addition to the one or more sensor layers.

Shoe insole 412 can also include a support layer 406. Support layer 406 can be located below sensor layer 404, as illustrated in FIG. 4A, or can be located anywhere else in shoe insole 412. Support layer 406 can be a structural component to an insole and can be made of natural or synthetic components such as cardboard, polyurethane, polymer plastics, carbon fiber, and the like. In certain embodiments the sensor layer 404 and the structural layer 406 may be one in the same layer.

Shoe insole 412 can also include a heel cup layer 408. Heel cup layer 408 can provide three-dimensional structure to insole 412. Heel cup layer 408 can also be used to provide a location to embed an electronics control module 410. Heel cup layer 408 can be made of natural or synthetic material similar to the support layer 406. In certain embodiments, heel cup layer 408 can be one in the same with the support layer 406. In addition, heel cup layer 408 can extend beyond the heel to, for example, the arch, where there is available three dimensional space to allow embedding an electronics unit 410.

Electronics unit 410 can comprise the electronic component 200, and can contain various sensors (202, 210) that do not need to be in direct contact with the foot-bed to collect their data. These sensors can include, for example, GPS sensors, acceleration sensors, etc. Electronics module 410 can also include the various processing 206, memory 208, battery 204, and communication 212 units. The various antenna 214, 216 may be included as part of the electronics unit 410 or, in certain embodiments of the present invention, embedded or co-existing with the sensors in the sensor layer 404.

While not shown, the electronics unit 410 can be electrically intercoupled with the sensor layer 404 even though those elements can be separated by other layers in the insole construction.

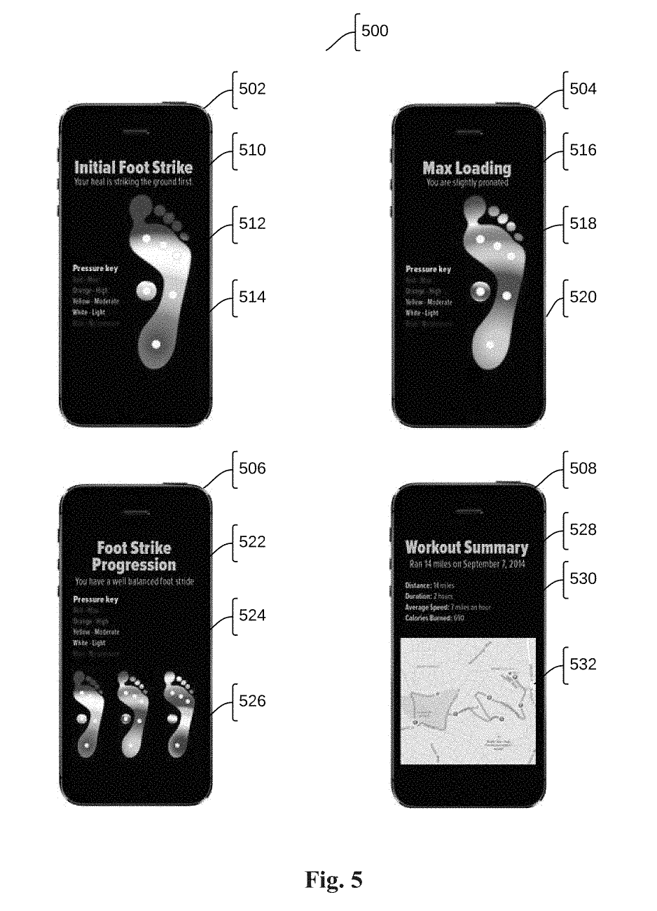

The data and information collected from the various sensors located in the shoe insole 412 can be relayed, via electronics unit 410/electronic component 200, to computer system 100, which can be a smartphone with suitable software applications as described above. As described above, and illustrated in FIG. 1, computer system 100 can include a display or user interface 118 and/or 122. Several possible screen displays from an exemplary user interface 500 are illustrated in FIG. 5. The user interface 500 can display output from the shoe insole 412 after monitoring biomechanics and motion. One possible screen 502 describes the specifics of an “initial foot strike” 510 and how the pressure is distributed across the foot under this circumstance 512. It includes a pressure key 514 describing the variations from high pressure to low pressure. In the pressure map 512, the key 514 indicates that the highest pressure is detected by the sensor in the heel, followed by the mid-foot and arch, with the least detected by the sensors distributed across the ball of the foot. A textual summary of this same visual information 512 can also be included in the description 510, such as “Your heel is striking the ground first.”

Another possible screen 504 shows the “max loading” 516 of the foot. The “max loading” 516 can also include a diagnostic textual summary such as “you are slightly pronated.” The textual summary 516 would summarize the visual display 518 of the pressure loading of the various sensors in the insole. In this example, as indicated by the pressure key 520, the highest pressure is experienced in the mid-foot, with slightly more pressure on the outside of the mid-foot than the inside (arch) of the mid-foot.

Yet another possible screen 506 shows the “foot strike progression” 522 as a series of foot pressure characteristics over time. A summary text 522 for this screen 506 may be “you have a well-balanced foot stride.” Again, a pressure key 524 is matched with a set of visual images 526 showing the sensor-detected pressures over time from a high pressure in the heel at first to a high mid-foot pressure, and followed by a high pressure region in the ball of the foot.

This combination of diagnostic screens 502, 504, 506, provides a small sample of diagnostic and diagnostic-related info′ nation that may be available to a sensor enabled insole. This type of information can be presented to the individual wearing the insole, or to their physician or physical therapist or coach. Similarly, the information may be stored and tracked over time allowing for understanding the progression of injury or recovery.

Still another possible screen 508 shows the workout summary 528 for the current use of the shoe insole 412. The workout summary 528 can have some descriptive text such as “ran 14 miles on Sep. 7, 2014.” This can be followed by more detailed information 530, for example, “Distance: 14 miles; Duration 2 hours; Average Speed: 7 miles per hour; Calories burned: 690.” This information can be directly measured with various sensors embedded in the insole, such as the distance and time (e.g. via the GPS sensor 210), while others may be derived or calculated measures, such as the average speed (distance divided by time) and calories burned (e.g. average speed with assumed amounts of energy used for running per time period, or perhaps more accurately by using average speed and average pressure to determine work effort). In addition, the GPS location can be plotted on a map 532 to help the user monitor or review progress.

Variations of the workout summary screen 508 could also include historical trending (e.g. your average speed over this course has increased by 0.2 mph in the last month), social competition information (e.g. you are in first place among your friends by 0.3 mph), or gamification elements (e.g. if you increase your speed by 0.1 mph you can set a course record). Similarly, social and gamification elements may be present in diagnostic screens 502, 504, 506.

The set of display screens 502, 504, 506, 508 are representative of the type of information that can be collected and displayed to the user or interested party (e.g. physician, coach, etc.), but are not intended to be exhaustive. One of ordinary skill in the art can readily identify alternate or additional information to display or to display information in a different manner. The provided screens 502, 504, 506, 508 are intended to only give a representative sample of the possible screens to indicate the possible scenarios where the data collected by the sensor-enabled insole may be used.

Additional understanding of the various usage scenarios are described in more detail in the following examples.

### Example 1—Orthopedics

Orthopedic uses can be described as the monitoring of bio-mechanical actions by layperson or medical professional for the purpose of prevention, diagnosis, monitoring and treatment.

### Example 1.1

Upon collection of the bio-mechanical actions, computer system 100, through component 118, can display a heat map of the foot at initial impact (e.g. FIG. 5, 502). Based upon the data, the system 100 can show a heat map within the first few milliseconds of the foot strike to show where on the foot the initial pressure is going and to determine if the user is heel striking, mid-sole striking or forefoot striking. Also display a message indicating to the user what type of foot strike they have (heel strike, midsole, etc)

### Example 1.2

Upon collection of the bio-mechanical actions, control system 100, through component 118, can display a heat map of the foot at max loading (e.g. FIG. 5, 504). Based upon the data, the system 100 can show a heat map at the point of max pressure measurement of all the sensors (when person's full body weight is on one foot) to show if the person is pronating or supinating and where on the foot most of the weight is distributed to.

### Example 1.3

Upon collection of the bio-mechanical actions, computer system 100, through component 118, can display a series of heat maps that show the foot pressure from impact through push off (e.g. FIG. 5, 506). Based upon the data, the system 100 can show a series of heat maps at specific intervals of the foot strike, from a minimum of 3 to 6 to 10 intervals, to show the characteristics of the foot strike and stride through initial impact and then push off.

### Example 1.4

Upon collection of the bio-mechanical actions, computer system 100, through component 118, can display a series of foot strike heat maps over a given time period. One possible example could include a series of heat maps that show the foot at max pressure over a given time (e.g. every 15 minutes over 2 hours). Another possible example could include a series of heat maps that show the foot at initial strike over a given time.

### Example 1.5

Upon collection of the bio-mechanical actions, computer system 100, through component 118, can display an analysis of how the relative pressure of specific zones of pressure on the foot changes over time with standard diagnosis of potential issues due to change in foot strike (e.g. increased pronation, increased impact pressure on heel, etc).

### Example 1.6

Upon collection of the bio-mechanical actions, computer system 100, through component 118, can display a message indicating what type of foot structure the user has (of the 3 typical foot type classifications: Neutral, Pronator, Supinator). Computer system 100 can then suggest a corrective course to improve a foot condition, monitor improvements, and provide continuous feedback to the user.

### Example 1.7

Upon collection of the bio-mechanical actions, computer system 100, through component 118, can display a heat map and message showing the difference in foot strike pattern and pressure between the left and right foot to help trace various health issue causes due to stride imbalance, leg length discrepancy, and other bio-mechanical imbalances caused by differences between the two foot strikes.

### Example 1.8

Upon collection of the bio-mechanical actions, computer system 100 can be used to send physical activity, cadence, foot strike data to a medical professional for monitoring of a patient. The medical professional can monitor the activity of a given patient through a World Wide Web or mobile application and provide advice to the patient through the application.

### Example 1.9

Based upon the bio-mechanical actions, computer system 100 can be used to send email or text alerts to medical professionals responsible for the care of patents using the sensor equipped insole. A software application can send alerts to medical professionals based upon specified parameters (e.g. no activity for a given period indicating a patient has fallen).

### Example 1.10

Upon collection of the bio-mechanical actions and in conjunction with known or algorithmically determined predictive criteria, shoe insole 412 and computer system 100 can be used identify conditions that can potentially lead to knee, back, leg, ankle, or foot injuries and warn the user or medical professional.

### Example 1.11

Upon collection of the bio-mechanical actions and using skeletal models, computer system 100 and shoe insole 412 can be used to calculate, and display or send to a medical professional the pressure on the skeletal system while running or performing other activities. Similarly, system 100 and insole 412 can be used to calculate pressure on vertebrae to alert the user or medical professional to possible injuries and suggest methods to avoid injury.

### Example 2—Lifestyle and Sports

These uses are described as monitoring, tracking and sharing physical activity by users.

### Example 2.1

Using a sensor-enabled shoe insole 412 to track distance, number of steps taken, or step speed for walking, running, or hiking activities. Insole 412, in connection with system 100 can calculate and display the number of steps for a given time period, or steps per mile/kilometer.

### Example 2.2

Using a sensor-enabled shoe insole 412 to track distance, pedal count, or cadence (number of pedal strokes per minute) for bicycling activity. A software application, as part of computer system 100, can calculate the pedal count per minute, per hour, and in total for a bicycle ride as well as the total distance traveled and the cadence for the ride and per mile/kilometer.

### Example 2.3

a display interface can show route and distance traveled on a map using GPS sensor data (e.g. FIG. 5, 508), which can allow users to save routes and track statistics like step count or pedal count, duration, or cadence for a given route. Software, which can be part of system 100 or standalone software can allow a breakdown of statistics per mile/kilometer and user defined segments on the map.

### Example 2.4

Using a sensor-enabled shoe insole 412 to track calories burned based on user entered statistics like height and weight and sensor data such as steps, cadence, distance, and specific activity. If so equipped, the pressure sensor data may be used to identify the user's weight in place of requiring the user to enter their own weight.

### Example 2.5

Using a sensor-enabled shoe insole 412 to provide bio-mechanical analysis to correct and improve technique. A software application, which can be part of component 200 and/or system 100 can provide cadence for running and biking to help athletes adjust cadence for higher efficiencies. The software application may also provide other measurements important to runners and bikers to improve form and technique such as foot strike duration, foot strike profile (heel strike verses midsole strike), pedal efficiency based on pressure of the foot in the bike shoe, or any other relevant measures and associated adjustments.

### Example 2.6

Using a sensor-enabled shoe insole 412 to calculate and display the weight of the person. System 100 can allow a user to track and display the weight of a person over time, or notify a healthcare professional or group of friends upon reaching specific goals. Alternately, computer system 100, through component 118 can display or notify the user or healthcare professional about weight gain. Similarly, system 100 can provide suggestions for weight management given monitored activity levels.

### Example 2.7

Allow users of a sensor-enabled shoe insole 412 to share goals and progress with other users of the insole as well as sharing with other non-users on social media sites.

### Example 2.8

Allow users of a sensor-enabled shoe insole 412 to set up shared goals (such as completing a marathon) and track progress of each user against the shared goal. Tracking can be real time to show progress of each user as the competition is happening.

### Example 2.9

Allow users of a sensor-enabled shoe insole 412 to sign up for open registration goals (anyone with the insole and software application can sign up) to compete against each other independent of location. Track and report upon progress of all users participating in the goal.

### Example 2.10

Track how long the shoe insole system sensors have been idle and send reminders and notifications via a mobile application to the user to ‘get in their shoes and move.’ Notifications may be configured to pertain to specific goals for activity, weight loss, training regimens, etc.

### Example 2.11

Using a combination of data collection and data pattern analysis, computer system 100, in connection with insole 412, can associate specific sensor identified movement patterns and associate to a movement classification (running, jump-roping, aerobics, etc). In this scenario the system learns differences in activity patterns across users so users do not need to specify activity types when tracking and reporting activities.

### Example 2.12

Allow users to set pulse rate goals for a specific work out, and use pulse rate sensors in the shoe insole to track real time pulse rate during exercise routine.

### Example 2.13

Using a sensor enabled shoe insole 412 to track and display foot pressure and balance characteristics during a golf swing to correct weight transfer issues during the swing to improve the golf swing.

### Example 2.14

Using GPS sensors (outside) or inertial sensors (indoors), track movement of a basketball player or multiple sensor equipped players on the court, helping with shot analysis (where people are shooting the ball from), defensive strategy and other team strategy.

### Example 2.15

Using a pulse sensor equipped insole 412, analyze user pulse rate including average pulse rate, max and min pulse rates for a given time period and for a day. With incorporated data storage, track the pulse rate over time (days, weeks, months) to determine improvements in physical fitness.

### Example 2.16

Using a sensor equipped insole 412, allow users to set step, pedal, calorie burn, and weight change goals, track progress against goals, show progress, and message when users complete goals.

### Example 3—Health and Medical

Use of the device and associated software application to monitor health conditions, can be used to help diagnose potential and present issues, monitor progress of corrective measures.

### Example 3.1

Utilizing a sensor equipped shoe insole 412 to provide analysis of walking stability and balance status of patients with central nervous system diseases. Information from the insole can be sent to medical professional for general monitoring and analysis.

### Example 3.2

Utilizing a pulse sensor equipped shoe insole 412 to send pulse rate data of a patient during exercise and resting states to a medical professional for analysis and general monitoring.

### Example 3.3

Utilizing a sensor equipped shoe insole 412 to provide full remote health and physical activity monitoring. Shoe insoles 412 allow a simple method to monitor patients' physical activity and send relevant updates to a health professional to aid remote monitoring in unassisted living situations.

### Example 3.4

Utilizing a sensor equipped shoe insole 412 to provide diabetic monitoring by monitoring physical activity of diabetes patents to determine when insulin should be taken and how much. Insulin frequency and dosage could be determined both by direct sensor detection of metabolic byproducts in sweat or other means, as well as indirect measures based upon GPS, inertial, or pressure monitoring of activity levels.

### Example 3.5

Utilizing a sensor equipped shoe insole 412 to provide Parkinson's disease monitoring, and to calculate drug dose needed based on physical activity, number of steps, and frequency of steps of patients. Identification of Parkinson tremor levels and balance upset can be monitored via pressure sensors in the insole.

### Example 3.6

Utilizing a sensor equipped shoe insole 412 to measure vascular stability and health based on the number of steps taken per day and general movement conditions.

### Example 3.7

Utilizing a sensor equipped shoe insole 412 to assist with post-stroke rehabilitation monitoring and direction. Health professionals can specify a recommended fitness routine and track the patient's daily progress remotely.

### Example 4—Big Data

Big data analysis of trends and conditions that lead up to injuries or sickness. Sensor-associated software application (used in connection with system 100 and insole 412) can provide analysis of a user's collected data against all other user data to detect trends or issues. This information can be used to predict injury and to recommend professional help.

### Example 4.1

Use foot strike and pressure data to determine if a user is at risk of foot, knee or back injury. An associated software application can compare pressure data of the user to diagnostic foot strike models developed in medical labs which characterize conditions which cause injury.

### Example 4.2

Use foot strike and pressure profiles to determine if a user is in need of orthotics or other medical help. An associated software application can compare foot strike and pressure data of a user to data from known orthotic candidates provided by medical research labs.

### Example 4.3

Use sensor equipped insole generated data to show how active a user is compared to other users based on age and gender. An associated software application can aggregate statistics from all users and provide comparative analysis for each user against groups of users with similar characteristics.

### Example 4.4

Use sensor equipped insole generated data from multiple users to compare cadence data to show a user how they compare with other runners or bikers. An associated software application can aggregate cadence statistics from all users and provide a comparative analysis for each user against groups of users with similar characteristics.

### Example 4.5

Use big data analysis techniques to determine the optimal steps or miles/kilometers for a given age group and gender to stay healthy and avoid injury. An associated software application can use recommendations and statistics from medical professionals, medical studies and medical groups (e.g. FDA, American Heart Association, etc.) to compare aggregate statistics from the user community and the user's specific statistics.

### Example 4.6

Pair a user account with social data (e.g. from Facebook, Twitter, etc.) to determine when users contract an illness or encounter other significant life events and associate movement behavior with specific life events. An associated software application can learn from these derivations and be able to predict events such as sickness, trauma, mental illness, and injury.

The examples provided above are not intended to be an exhaustive explanation of each possible operation of the systems and methods described herein, and the various embodiments are not limited to any example described above.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required.” Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A wireless monitoring and tracking shoe insole device for monitoring biomechanics and motion, the insole device comprising:
a removable shoe insole portion having at least one layer;
a plurality of strategically positioned sensors disposed within the insole portion; and
an electronic component disposed within the insole portion, the electronic component intercoupled to the plurality of sensors and configured for collecting, storing and relaying sensor data from the plurality of sensors.

2. The insole device of claim 1 wherein the insole portion comprises:
a top cover layer;
a sensor layer;
a support layer; and
a heel cup layer.

3. The insole device of claim 2 wherein the sensor layer is disposed between two other layers of the insole portion.

4. The insole device of claim 2 wherein the electronic component is disposed within the heel cup layer.

5. The insole device of claim 1 wherein the plurality of sensors comprises:
at least one pressure sensor;
at least one acceleration sensor; and
at least one GPS sensor.

6. The insole device of claim 5 wherein the plurality of sensors further comprises:
at least one rotation rate sensor; and
at least one inertial sensor.

7. The insole device of claim 5 wherein a plurality of pressure sensors and a plurality of acceleration sensors are positioned horizontally across the at least one layer of the insole portion.

8. The insole device of claim 1 wherein the electronic component is wirelessly connected to an external computer system configured for collecting, processing, storing, displaying and relaying sensor data transmitted from the electronic component of the insole device.

9. The insole device of claim 8 wherein the computer system includes a user interface configured for displaying sensor data.

10. A method of monitoring biomechanics and motion information through a sensor equipped shoe insole having a plurality of sensors coupled to an electronic component configured for collecting and transmitting sensor data collected from the plurality of sensors, the method comprising the steps of:
collecting first sensor data from a first sensor and storing the collected first sensor data in a memory contained within the electronic component;
collecting second sensor data from a second sensor and storing the collected second sensor data in the memory;
collecting third sensor data from a third sensor and storing the collected third sensor data in the memory;
repeating collection and storage of first, second and third sensor data for a plurality of iterations;
transmitting the collected sensor data to an external wirelessly connected system;
examining, parsing and configuring the collected sensor data through instructions provided in the external wirelessly connected system; and
displaying the configured sensor data through a user interface provided on the external wirelessly connected system.

11. The method of claim 10 wherein the plurality of sensors includes pressure, acceleration, and position sensors.

12. The method of claim 11 wherein the plurality of sensors further includes rotation rate sensors and inertial sensors.

13. The method of claim 12 wherein the steps of collecting first, second, and third sensor data from the first, second, and third sensors are performed for each of the pressure sensors, the acceleration sensors, the rotation rate sensors and the position sensors.

14. The method of claim 10 further comprising the step of collecting sensor data from a long-cycle sensor for GPS position after the plurality of iterations have been completed.

15. A combination sensor-equipped shoe insole and external software application comprising:
a shoe insole having a plurality of layers, a plurality of sensors positioned within the plurality of layers, and an electronic component coupled to the plurality of sensors for collecting, storing and transmitting sensor data; and
an application executed on an external computer system, the system having a processor and memory for storing and compiling user information from the sensor data and a user interface for displaying the user information;
wherein the shoe insole collects pressure, acceleration, and route tracking data from the plurality of sensors without being synced to the external computer system.

16. The combination of claim 15 wherein the shoe insole further collects rotation rate data and inertial data in three axes from the plurality of sensors without being synced to the external computer system.

17. The combination of claim 15 wherein application is configured for displaying on the user interface diagnostic information on orthopedic metrics including foot pronation, supination, and heel strike based on the sensor data collected from the shoe insole.

18. The combination of claim 15 wherein the application is configured for displaying on the user interface a plurality of fitness metrics including cadence, distance, calories burned, and pulse rate based on the sensor data collected from the shoe insole.

19. The combination of claim 15 wherein the application is configured for transmitting a first user's sensor data to an external collective computer system for aggregating a plurality of user's sensor data.

20. The combination of claim 15 wherein the application is configured for enabling multiple users to view sensor data from other users.
